(19) United States
(12) Patent Application Publication
(10) Pub. No.: US 2025/0248936 A1
(43) Pub. Date: Aug. 7, 2025

(54) COATED CONCENTRATED COMPOSITION AND USES THEREOF, USEFUL FOR CONTROLLING GASTROINTESTINAL DISEASES IN BIRDS AND IMPROVING PRODUCTIVE PARAMETERS

(71) Applicant: PLANTAE LABS SPA, Santiago (CL)

(72) Inventors: Rosa NAVARRO LISBOA, Santiago (CL); Sebastian DECAP SWINBURN, Santiago (CL); Hans KONSENS CAMPOSANO, Santiago (CL)

(73) Assignee: PLANTAE LABS SPA, Santigo (CL)

(21) Appl. No.: 18/855,168

(22) PCT Filed: Apr. 6, 2023
(86) PCT No.: PCT/CL2023/050033

§ 371 Date: Oct. 8, 2024

Related U.S. Application Data
(60) Provisional application No. 63/328,845, filed on Apr. 8, 2022.

(51) Int. Cl. A61K 9/107; A61K 9/00; A61K 36/53; A61K 47/10; A61K 47/22; A61P 33/02

(52) U.S. Cl. CPC A61K 9/1075; A61K 36/53; A61K 47/10; A61K 47/22; A61P 33/02; A61K 9/0053

(57) ABSTRACT

The present invention discloses a coated concentrated composition which allows the controlled release of an extract rich in saponins and polyphenols, which is used for controlling gastrointestinal diseases in birds and improving productive parameters.

### FIELD OF THE INVENTION

The present invention relates to a concentrated, coated composition allowing controlled release of a saponin and polyphenol-rich extract rich, which is used to control gastrointestinal diseases in monogastric animals and to improve productive parameters.

### STATE OF THE ART

Digestive tract diseases affect all kinds of birds, especially grain-fed chickens and turkeys. Many of these diseases are caused by various pathogens, accompanied by nutritional imbalances that can lead to clinical or subclinical symptoms, which are related to such factors as: quantity of oocysts or pathogenic load ingested at once, with the animal's age when it is infected, with the species' pathogenicity, and prevention control measures as implemented by the farms. These diseases can cause great economic losses due to decreased feed conversion rate, weight loss, dehydration, secondary infections, poor absorption of specific nutrients, exacerbation of other diseases and can even cause the death of newborn birds. High-density animal husbandry has increased exposure to these parasitic diseases. These diseases, among others, include:
1) Coccidiosis: this disease is caused by intracellular protozoan parasites of the Eimeria species, Eimiridae family, Eucoccidiorida order, and phylum Apicomplexa. Eimeria infects the digestive tract, mainly of chickens and turkeys, which infection spreads from bird to bird. This infection depends on the survival of oocysts present in soil, feces or contaminated water, which are ingested by other susceptible birds. The species most commonly present in chickens are: Eimeria acervulina, Eimeria tenella, Eimeria necatrix, Eimeria brunetti, and Eimeria maxima, while, in turkeys are Eimeria adenoeides, Eimeria gallopavonis and Eimeria meleagrimitis. Eimeria tenella is one of the most common and pathogenic species, causing high mortality due to the extensive damage it causes in the digestive tract of chickens. On the other hand, the different species of Eimeria can cause alterations in epithelial cells and excretion of large quantities of fluids and mucosa. However, only E. tenella and E. necatrix cause significant blood loss.
2) Necrotic enteritis: This is a disease having a great economic impact on the grain-fed chicken industry mainly affecting grain-fed chickens. It is caused by Clostridium perfringens, a gram-positive spore-forming anaerobe, which causes damage to the intestinal mucosa, which causes the bacteria to reach the bile-duct and the bloodstream. This leads to production losses due to poor digestion and absorption, lower weight gain and a higher feed conversion rate.
3) Histomoniasis: this is another very complex disease caused by protozoan parasites called Histomona meleagridis. H. meleagridis is a unicellular parasite of the Parabasalia phylum, Tritrichomonadea class, which mainly affects birds. Turkeys are especially susceptible to H. meleagridis infection with lesions in the cecum and liver. However, other poultry can also be infected and act as hosts, such as partridges, quail, pheasants and chickens, affecting the cecum where the cecumis mostly affected. Once inside the intestine, these parasites migrate to the cecum, replicating and degrading the caecal lining. The transmission of this disease is caused by:
1) ingestion of the adult common cecal worm (Heterakis gallinarum) or its eggs infected with H. meleagridis;
2) transmission by ingesting infected material that is contaminated with protozoan) such as feces or invertebrates, although some invertebrates can serve as intermediate mechanical vectors of infected heterokid eggs;
3) direct transmission of H. meleagridis through cloacal drink and reverse peristaltic movement towards the ventilation region;
4) transmission of infected eggs by carriers. Chickens are considered potentially resistant to Histomoniasis, by acting as asymptomatic carriers and reservoirs of eggs infected with H. meleagridis. The prohibition of many of the drugs used to combat the disease, Histomoniasis is gaining greater relevance in several countries, reason by which it turns out to be interesting to apply control systems and treatments for preventing this disease.

For years, many of these diseases have been controlled by using chemicals, antibiotics, and/or ionophores. However, its prolonged use has started to cause concern among consumers due to the residues that can remain in the flesh (their toxicity and carcinogenic effects. In addition, current animal welfare, feed safety, reduction of environmental impacts, and prohibition of the use of growth-promoting antibiotics are factors leading to the search of new solutions for this market. Currently, 80% of the antibiotics sold in the United States are used in animals. This shows that the industry is facing a major problem in fighting off infectious diseases.

Therefore, the main problem lies in the adverse effects caused by the current solutions for controlling gastrointestinal pathogens in birds and the lack of natural solutions that do not degrade at intestinal level, having controlled release and a proven effect at the pathogens' action site.

The present invention relates to the development of a natural product showing improvements as to alternatives currently existing in the state of the art, since it has a proven effect at the action site and is resistant to passing through the digestive tract. The product comprises a natural, saponin-rich extract, made up of a triterpenoid and/or steroid saponin-rich mixture and phenolic compounds, which is coated with polymeric multilayers and fortified with oregano essential oil. The natural saponin-rich extract is encapsulated by using a mixture of natural fibers and digestive tract-resistant polymers, forming a cross-link allowing a controlled release in the large intestine and cecum, and which, depending on the disease, may or may not be enhanced with oregano essential oil.

The present invention discloses a natural product showing improvements as compared to the alternatives existing in the state of the art, since it has a controlled release effect at the action site and is highly resistant to pH conditions and digestive tract enzymes.

### BRIEF DESCRIPTION OF THE INVENTION

The present invention relates to the development of a natural product showing improvements with respect to the alternatives existing in the state of the art, since it has a proven effect at the action site and is resistant to the digestive tract. The product comprises a natural saponin-rich extract, made up of a (mixture rich in triterpene and/or steroidal saponins and phenolic compounds, which is coated with polymeric multilayers and enhanced with oregano essential oil.

### DETAILED DESCRIPTION OF THE INVENTION

The present invention relates to the use of a mixture of natural fibers allowing for controlled release of a saponin-rich extract and polyphenols that is used for controlling gastrointestinal diseases in animals and improving productive parameters, and which can be enriched with oregano essential oil for a greater effect on pathogen control.

One embodiment of the present invention discloses a coated concentrated composition for controlling digestive tract diseases in animals comprising:
between 0.1-95% w/w of saponin-rich extract (or pulverized wood);
between 0.1-35% w/w of fiber; and
between 0.1-35% w/w of polymer; and
water cc. s.q. for 100%,

In one embodiment of the present invention, the coated concentrated composition has a particle size between 1-10000 μm.

In one of the embodiments of the present invention, the composition optionally comprises active ingredients transmitted through micro-emulsion systems.

In one of the embodiments of the present invention, the composition comprises further active ingredients transmitted through micro-emulsion systems, which may include:
optionally, between 5-30% w/w of an essential vegetal oil;
optionally, between 5-30% w/w of a co-surfactant;
optionally, between 0.1-5% w/w of a natural vegetal antioxidant-rich extract, and/or
optionally, between 0-20% w/w of an emulsifier.

In one of the embodiments of the present invention, the saponin-rich extract of the composition is selected from quillay, yucca, quinoa, fenugreek, garlic, (fennel, tea, ginseng, alfalfa, oats, fique, chocho, agave, black beans or combinations thereof.

In one of the embodiments of the present invention, the fiber of the composition is selected from carboximetil cellulose, guar gum, pectin, xanthan gum, inulin, amylose, dextran, chitosan or combinations thereof.

In one embodiment of the present invention, the polymer of the composition is selected from modified starch, starch, maltodextrin, guar gum, corn syrup, agar, proteins, alginate, or combinations thereof.

In one of the embodiments of the present invention, the vegetal essential oil of the composition is selected from oregano essential oil, thyme, lemongrass, eucalyptus, orange, clove, bayleaf, lemon, cinnamon, mustard, savory, copaiba, or combinations thereof.

In one embodiment of the present invention, the antioxidant agent of the composition is selected from (rosemary, tocopherol, curcumin, astaxanthin, or combinations thereof.

In one embodiment of the present invention, the co-solvent agent of the composition is selected from polyols, glycerin, propylene glycol, or combinations thereof.

In one of the embodiments of the present invention, the emulsifying agent of the composition is selected from saponins, lecithins, polar lipids, or combinations thereof.

In one of the embodiments of the present invention, the composition is active against digestive tract diseases in animals, as selected from the Eimeria, Histomonas, Staphylococcus, Escherichia, Pseudomonas, Streptococcus, Clostridium genus, or other pathogens causing diseases in the cecum or intestinal diseases of interest in animal health, or combinations thereof.

In one of the embodiments of the present invention, the composition may be orally administered to the animal via pills, capsules, injection, in feed, powdered, or liquid state, or combinations thereof.

One of the embodiments of the present invention discloses the use of the coated concentrated composition which is useful to prepare an additive formulated to control gastrointestinal infections in animals and to improve intestinal health in animals.

In one of the embodiments of the present invention, it is useful for preparing an additive for controlling gastrointestinal infections under in vitro and in vivo conditions.

In one of the embodiments of the present invention, the infections are caused by microorganisms selected from the Eimeria, Histomonas, Staphylococcus, Escherichia, Pseudomonas, Streptococcus genuses and other microorganisms of veterinary relevance.

One of the embodiments of the present invention discloses a method for preparing a liquid, coated, concentrated composition for controlling digestive tract diseases in animals comprising the following steps:
a) mixing the saponin-rich extract with oregano that is transmitted by using micro-emulsion technology;
b) The micro-emulsions are prepared as follows:
   i) Preparing the continuous phase by 200-5000 rpm mechanical stirring, containing a saponin-rich extract as emulsifiers, glycerin as co-solvents, polymers as stabilizer, and water as solvent;
   ii) Preparing the lipidic phase containing essential oil and a mixture of natural antioxidants. Trying to keep the emulsifying ratio: natural oil ratio at 1:2-1:10;
   iii) Mixing both phases via a 3000-8000 rpm via a high shear homogenization process for 10 minutes to form the pre-emulsion; and
   iv) Subsequently, homogenizing using high pressure equipment at 300-500 psi, in two stages.

One of the embodiments of the present invention discloses a procedure for preparing a powder-coated concentrated composition for controlling digestive tract diseases in animals, which comprises the following steps:
a) mixing the saponin-rich extract and the natural fiber by 100-500 rpm mechanical stirring and at a temperature between 40° C. (104° F.)-80° C. (176° F.) until obtaining a homogeneous mixture;
b) setting pH to reach lower values according to the pKa of the fiber used for gelation and by trapping of the saponins in the grid, through interactions between them and the fiber;
c) adding the fiber and saponins at a 1:1 ratio, more specifically 1:10, more specifically 1:50, which is key for the formation of the particles;
d) adding the polymer as a drying agent under constant stirring;
d) increasing the pH to promote capsule formation and good drying performance;
e) keeping constant the temperature of the mixture to preserve the necessary fluidity of the mixture for the drying procedure; and
f) drying the mixture by atomization, keeping a constant delta between the mixture's temperature of and the dryer's input temperature with values ranging from 50° C. (122° F.-130° C. (251.6° F.).

### EXAMPLES

### Liquid Trials for Coccidiosis Control:

The present study uses a total of 576 male Cobb 500 chickens. The birds were reared from 0 to 28 days of age in cages: 72 cages holding 8 birds/cage, and 9 cages/treatment. The birds were randomly assigned to the treatments. The birds were fed corn-soybean meal as a lipid source. The feed and water were provided ad libitum. The treatments are shown in Table 1. Except for Maxiban (MAX) administered through the feed, all other additives were administered through drinking water on a continuous basis. The water additives comprised Quillaia saponaria extract or Q. saponaria extract, combined with oregano oil. No birds were replaced during the study.

TABLE 1
Experimental design.

| | | | E. maxima challenge |
|---|---|---|---|
| Treatment | | Inclusion | (Day 14) |
| Control | CON | — | — |
| Control/challenge | POS | — | Yes |
| Maxiban | MAX | | Yes |
| Quillaia - oregano | QOE1 | 0.12 g/l | Yes |
| emulsion | | | |
| Quillaia - oregano | QOE2 | 0.30 g/l | Yes |
| emulsion | | | |
| Quillaia - liquid | QLS | 0.06 g/l | Yes |
| solution | | | |

On day 14, all the birds (except for CON) were orally administered a mixed inoculum of E. acervulina, E. maxima and E. tenella diluted in 1 mL (p.o.). The quantity of inoculum is intended to produce a moderate challenge. All the birds in the CON group 5 were given 1 mL of distilled water.

TABLE 2
Formulations.

Liquid formulation

| | Example 1 (QOE1) | Example 2 (QOE2) |
|---|---|---|
| Quillaia extract | 2.3-6.8 | 6-14.3 |
| (8.5% *saponins) | | |
| Water | 96-87.8 | 78.8-50 |
| Oregano oil | 0.5-1.4 | 3.3-7.8 |
| Tocopherol | 0.2-0.5 | 0.55-1.3 |
| Glycerin | 0.8-2.3 | 10.7-25.4 |
| Fiber | 0.5-1.4 | 0.55-1.3 |

Sample description: QOE1 is a mixture of Quillaia extract, polymer and oregano emulsion that is mixed by homogenization at 3000 rpm for 2 hours. QOE2 is a mixture of Quillaia extract, oregano oil, tocopherol, glycerin and polymer as mixed via a high shear homogenization process at 3000-5000 rpm, for 10 minutes, followed by high pressure at 300-500 psi in two stages. QLS is a 6% saponin Quillaia water extract.

On day 21, 3 birds/cage were slaughtered and lesion scores were assessed by using a 0-3 scoring scale, 0 being normal, and 3 being the most severe. Feed intake (FI), body weight gain (BWG) and feed conversion ratio (FCR) were recorded every 7 days. Blood samples were taken for fluorescein isothiocyanate-dextran (FITC-d) analysis on day 21 (2 birds/cage). For this purpose, each bird is administered 1 mL of FITC-D solution (2.2 mg/mL concentration) by gavage, each treatment being started 2 hours following said lavage. Finally, a 1 mL blood same is taken, without coagulant, and stored at 4° C. (39.2° F.) until proper analysis.

### Results

For the 0 to 28 day period, BWG and FCR in the POS treatment were significantly different as compared to the CON treatment; 24% FCR increase, and 34% BWG reduction (Table 3). Compared to untreated/infected (POS) birds, QLS treatment showed a slight FCR reduction (3%) and 13% BWG increase. BWG and FCR did not differ significantly between MAX, QOE1, QOE2 and QLS treatments. The challenge had a significant impact on productive parameters as observed by BWG and FCR with most significantly different values in POS versus CON over the 14-28 day period; birds on POS underwent a 35% BWG reduction and a 25% FCR increase. When comparing to untreated/infected birds (POR), treatments QOE2, QLS, and MAX had the lowest FCR and highest BWG of all treatments.

TABLE 3
0-28-day- and 14-28-day-productive parameters

| | Day 0-28 | | | Day 14-28 | | |
|---|---|---|---|---|---|---|
| Treat- | FI (kg/ | BWG | | FI (kg/ | BWG | |
| ment | cage) | (kg) | FCR | cage) | (kg) | FCR |
| CON | 9.896a | 0.897a | 1.541b | 6.392a | 0.702a | 1.418d |
| POS | 9.201ab | 0.663c | 1.866a | 5.573b | 0.464c | 1.873a |
| MÁX. | 9.320ab | 0.783b | 1.765a | 5.591b | 0.586b | 1.655c |
| QOE1 | 8.786b | 0.686bc | 1.903a | 5.156b | 0.509bc | 1.783ab |
| QOE2 | 9.064ab | 0.733bc | 1.822a | 5.420b | 0.552bc | 1.681 a.C. |
| QLS | 8.909b | 0.758bc | 1.809a | 5.280b | 0.563b | 1.734ab |

Within a column, the values with different letters are significantly different (P < 0.05).

All the natural treatments (QOE1, QOE2 and QLS) had a significant impact on Eimeria lesion score, better than MAX treatments. Compared to untreated/infected birds (POS), QOE2 had lower E. maxima and E. Tenella lesion score values, followed by QLS and QOE1, showing that they can reach the midgut and cecum, being better than MAX.

TABLE 4
Lesion score for different Eimerias.

| | Eimeria | Eimeria | Eimeria | |
|---|---|---|---|---|
| Treatment | acervulina | maxima | tenella | Mean |
| CON | 0.00c | 0.00c | 0.00d | 0.00d |
| POS | 2.82a | 2.18a | 2.44a | 2.48a |
| MAX | 2.00b | 2.20a | 1.91b | 2.04b |
| QOE1 | 2.07b | 1.56b | 1.73bc | 1.78c |
| QOE2 | 2.13b | 1.53b | 1.53bc | 1.73c |
| QLS | 1.98b | 1.58b | 1.71bc | 1.76c |

For the study, all the treatments, on average, had fewer oocysts/gram than POS. Treatments QOE1 and QOE2 had lower Oocysts/gram for E. maxima and E. tenella than POS and MAX.

TABLE 5
Quantification of *Oocysts/gram for different Eimerias.

| | Eimeria | Eimeria | Eimeria | |
|---|---|---|---|---|
| Treatment | acervulina | maxima | tenella | Total |
| CON | 0b | 0c | 0b | 0b |
| POS | 14207a | 5291ab | 10954a | 30452a |
| MAX | 6500ab | 8256a | 5336ab | 20092a |
| QOE1 | 10679ab | 4965ab | 4498ab | 20143a |
| QOE2 | 7485ab | 4521b | 4802ab | 16809a |
| QLS | 9427ab | 4461b | 9301a | 23189a |

In conclusion, it is shown that there was a clear impact of the Eimeria challenge on the birds' productive parameters; a 26% BWG reduction was observed in infected birds (POS) as compared to uninfected birds (CON) during the 28-day period. The administration of the Quillaia extract and Oregano oil (QOE1, QOE2 and QLS) combination did not differ significantly between MAX as regards such parameters as BWG and FCR. All the natural treatments (QOE1, QOE2 and QLS) had a significant impact on Eimeria lesion score values, being the best as treatments with respect to the use of MAX.

With respect to untreated/infected (POS) birds, QOE2 showed the lowest lesion drop of E. maxima and E. Tenella, followed by QLS and QOE1.

### Example 1. Powdered Solution to Control Coccidiosis

On occlusion day, male grain-fed chickens (Cobb X) from Cobb-Vantress, Cleveland, GA, were used in the study. A total of 360 chickens were randomly selected, group held, and caged in the study. Birds were bred from 0 to 28 days of age in cages, with 10 birds/cage and 6 cages/treatment. No birds were replaced during the course of the study. On day 14 of the study, all birds, except for CON, were orally administered diluted E. tenella inoculum in a quantity of 1 mL (po). The inoculum level was intended to produce a high infection challenge (20% mortality). All the birds in the CON group were administered 1 mL of distilled water.

TABLE 6
Experimental design

| Treatment | Inclusion | E. Tenella Challenge (day 14) |
|---|---|---|
| CON | — | — |
| POS | — | Yes |
| QSC | 30 ppm | Yes |
| QHS-LD | 36 ppm | Yes |
| QHS-HD | 45 ppm | Yes |
| QSC-C | 250 ppm | Yes |

QSC and QHS contain the same quantity of saponins. Low QHS-LD dose; High QHS-HD dosage.

On day 21, 3 birds/cage were slaughtered and the lesion score was assessed by using a 0-3 scoring scale, 0 being normal, and 3 being the most severe. Feed intake (FI), body weight gain (BWG), and feed conversion ratio (FCR) were recorded every 7 days.

TABLE 7
Trials.

| | Example 1 | Example 2 | Example 3 |
|---|---|---|---|
| | (QSC) | (QHS) | (QSC-C) |
| Quillaia Extract (10.5% saponins) | 30-55 | 25-50 | — |
| Fiber ((inulin, pectine, amylose, | 2-10 | 2-10 | — |
| dextran, chitosan, guar gum, | | | |
| xanthan gum) | | | |
| Polymer as an astringent (starch, | 2-5 | 4-12 | — |
| maltodextrin, guar gum, corn syrup, | | | |
| agar, carboxymethylcellulose) | | | |
| Water | 66-30 | 69-28 | — |
| SC | — | — | 3-20 |
| Carrier (zeolites, calcium | — | — | 97-80 |
| carbonate, rice bark). | | | |

Sample description: QSC is a mixture of Quillaia extract and natural fiber that is mixed, via homogenization, at 200 rpm for 2 hours at 40° C.-80° C. (104° F.-176° F.). The pH is set to reach lower values according to the fibers' pka, which promotes fiber expansion and allows to capture/fix saponin molecules, forming mixed soluble compounds triggered by the sugars of the adsorbed saponins. Subsequently, a polymer, that is used which is used to facilitate the formation of a film on the surface of the drops trapping the remaining water and facilitating the drying process, is added. Finally, the mixture's pH is set to promote the formation of compounds and improve drying performance. The mixture must be put inside the dryer at a suitable temperature thus allowing fluidity and preventing caramelization in the sprayer.

QSC-C is a product containing QSC product and co-formulants to set the saponin at the commercial dosage. This was tested to assess whether the co-formulants affect the efficacy of the products.

QHS is a Quillaia extract containing different polymer sources used to create a stable grid allowing it to pass through the intestinal tract and reach the midgut and cecum. Firstly, fibers are added, the pH being set to reach lower values according to the fibers' pka, which promotes fiber expansion and allows the capture/fixation of saponin molecules, forming mixed soluble compounds triggered by the sugars of the adsorbed saponins. Subsequently, different low-solubility and intestinal degradation polymers are added, which are basically used to generate a more resistant film on the particles' surface during the drying process.

Drying conditions are adapted to the polymer matrix where the most important variables are 150° C.-200° C. (302° F.-392° F.) input temperature, 40%-70% atomization, and 10%-30% feed flow.

### Results

For the 0 to 21-day period, POS treatment showed a significant BWG reduction (15%) as compared to the CON group, and a FCR 14% increase (Table 6). When comparing with untreated/infected birds (POS), QHS-HD showed a slight FCR reduction (6%) and a 5% BWG increase, where it was similar to the QSC and QHS-LD treatments. BWG and FCR did not significantly differ between the QSC, QHS-LD, and QHS-HD groups. The Eimeria challenge had a significant impact on productive parameters, as observed by the lowest BWG (32% reduction) and FCR (27% increase) in the POS versus CON group over the 14- to 21-day period. When comparing to untreated/infected (POS) birds, QSC showed a significant FCR reduction (9.1%) and BWG increase (11%), respectively, and these values were of the same magnitude as QHS-HD. Both treatments, QSC and QHS-HD, were similar to QSC-C, which demonstrates that the agents used as co-formulants (calcium carbonate, zeolites, etc.) do not affect the functionality of the encapsulated additive. QHS-LD did not significantly differ with respect to QHS-HD and QSC treatments, but, numerically, its performance was slightly lower. Feed intake was slightly different between treatments throughout the study and the period during the challenge (14-20 days).

TABLE 8
Productive parameters 0-21/day and
14-21/day productive parameters.

| | Day 0-21 | | | Day 14-21 | | |
|---|---|---|---|---|---|---|
| | FI | BWG | | FI | BWG | |
| Treatment | (kg/cage) | (kg) | FCR | (kg/cage) | (kg) | FCR |
| ESTAFA | 6.391b | 0.569a | 1.466c | 3.172ab | 0.330a | 1.273c |
| TPV | 6.497ab | 0.484b | 1.683a | 3.128b | 0.225d | 1.739a |
| QSC | 6.536ab | 0.515ab | 1.599b | 3.168ab | 0.253bc | 1.580b |
| QHS-LD | 6.341b | 0.490b | 1.628ab | 3.115b | 0.243cd | 1.611b |
| QHS HD | 6.640ab | 0.531ab | 1.588b | 3.230ab | 0.252bc | 1.616b |
| QSC-C | 6.884a | 0.549a | 1.576b | 3.443a | 0.272b | 1.583b |

Within a column, the values with different letters are significantly different (P < 0.05).

The QHS-LD treatment showed a significant reduction (31%) in lesion score values and a 48% reduction in oocysts/gram as compared to the POS group, and was slightly better as compared to the other treatments. This could be the best treatment to reach the cecum, where the site of E. tenella infection is located, indicating that this technology is highly efficient at treating gastrointestinal diseases in birds.

main advantages of powdered products are stability against external factors, the ability to modulate release according to the required action site, the ability to be mixed, and the option of being administering in small doses.

TABLE 9
Lesion score and oocysts/gram of the different treatments

Eimeria tenella

| Treatment | Lesion score | Oocysts/Grams |
|---|---|---|
| CON | 0.0c | 0.0c |
| POS | 3.2a | 19720^{a} |
| QSC | 2.1c | 13093b |
| QHS-LD | 2.2c | 10229b |
| QHS HD | 2.5b | 11541b |
| QSC-C | 2.3bc | 11496b |

In conclusion, it is demonstrated that there was a clear impact of the E. tenella challenge on the productive parameters for birds; a 26% BWG reduction was observed in exposed birds (POS) as compared to unexposed control birds (CON) during the 21-day period. The administration of Quillaia extract, as mixed with natural polymers and fiber, specifically promotes E. tenella control. The addition of co-formulants did not have a significant impact on QSC's efficacy, thus demonstrating that the product does not lose efficacy at controlling gastrointestinal diseases.

### Example 2 Powdered Solution for Controlling Coccidiosis

A total of 270 male Cobb 500 chickens were used in the present study. The birds were randomly assigned to treatments. The birds were fed corn-soybean meal as a lipid source. Feed and water were provided ad libitum. All the treatments were administered, on a continuing basis, via feed. No birds were replaced during the study. On day 14, all birds, except for CON, were orally administered diluted inoculum of E. tenella, E. maxima, and E. acervulina in a quantity of 1 mL (p.o.). The inoculum level was intended to produce a high infection challenge. All the birds in the CON group were administered 1 ml of distilled water. On day 21, 3 birds/cage were slaughtered and the necrotic enteritis lesion score was were evaluated by using a 0 to 3 scoring scale, 0 being normal, and 3 being the most severe. Feed intake (FI), body weight gain (BWG) and feed conversion ratio (FCR) were recorded every 7 days.

TABLE 10
Experimental design

| | | | Number of | Number of |
|---|---|---|---|---|
| Treatment | Infected | Dose | cages | chicken/cage |
| CON | No | — | 9 | 6 |
| POS | Yes | — | 9 | 6 |
| MAX | Yes | 72 ppm | 9 | 6 |
| QS15 | Yes | 30 ppm | 9 | 6 |
| QSC | Yes | 30 ppm | 9 | 6 |

QS15 is a mixture of saponin-rich extract to which a highly soluble polymer is added as an astringent that facilitates optimal extract drying. The drying conditions are input temperature of 110° C.-170° C. (230° F.-338° F.), 50%-90% atomization, and 20%-40% feed flow.

QSC is a mixture of Quillaia extract and natural fiber that is mixed by homogenization at 200 rpm for 2 hours, at 40° C.-80° C. (194° F.-176° F.). pH is set to reach lower values according to the fiber's pka, which promotes fiber expansion and allows the capture/fix saponin molecules, forming mixed soluble compounds triggered by the sugars of the adsorbed saponins. Subsequently, a polymer, which is used to facilitate the formation of a film on the surface of the drops thus trapping the remaining water and facilitating the drying process, is added. Finally, the pH of the mixture is set to promote the formation of compounds and improve drying performance. The mixture must be put in the dryer at a suitable temperature thus allowing fluidity and preventing the formation of caramelized particles in the sprayer.

As shown in Table 11, the coccidiosis challenge had a significant impact on performance, as observed by the differences in BWG and FCR values in the POS group versus CON group, for the 14 to 21-day period; the birds in the POS group showed a 64% BWG reduction, and a 63% FCR increase versus CON. As compared to untreated/infected birds (POS), the QS15 group showed a slight increase (10%) and reduction (17%) in FCR and BWG, respectively. QS15 and QSC showed a significantly lower FCR than the POS and MAX groups. Feed intake slightly differed between treatments in the overall study and the period during the challenge (14-20 days), with the CON and POS treatments showing the highest values.

TABLE 11
Productive Parameters - 0-20 days and 14-20 days

| | FI | FI | FCR | FCR | BGW (kg) | BGW (kg) |
|---|---|---|---|---|---|---|
| Treatment | D 0-20 | D 14-20 | D 0-20 | D 14-20 | D 0-20 | D 14-20 |
| CON | 4.715ab | 2.941ab | 1.271 | 1.353e | 0.473a | 0.272a |
| POS | 4.882a | 2.946a | 1.649a | 2.147a | 0.391cd | 0.172d |
| MAX | 4.702ab | 2.703abcd | 1.482bc | 2.81a | 0.413bcd | 0.199bc |
| QS15 | 4.756ab | 2.798abc | 1.514abc | 1.832bc | 0.402bcd | 0.191bcd |
| QSC | 4.729ab | 2.692abcd | 1.380cd | 1.665d | 0.437ab | 0.203b |

As shown in Table 12, the Max and QS15 treatments had lower Oocysts/gram for all the Eimerias than the POS group. The relevant aspect was the fact that the treatment with QSC showed a significant reduction as compared to the POS, MAX and QS15 groups, and unlike the other treatments, QSC showed a greater decrease in E. maxima and E. Tenella, reason by which it may be deduced that this technology is specific to the release at the end of the digestive tract.

TABLE 12
(Oocyst/gram quantification for the different Eimerias

| | Oocyst/grams | | |
|---|---|---|---|
| | Eimeria | Eimeria | Eimeria |
| Treatment | acervulina | maxima | tenella |
| CON | 0c | 0b | 0c |
| POS | 84659a | 11089ab | 9888a |
| MAX | 16692bc | 16658a | 2485bc |
| QS15 | 39236b | 16333a | 4177abc |
| QSC | 34659b | 3135b | 2710bc |

### Example. Liquid Products for Controlling Necrotic Enteritis

A total of 576 male Cobb 500 chickens were used in this study. The birds were bred, from 0 to 28 days of age, in cages, in 72 cages, each cage holding 8 birds/cage and 9 cages/treatment. The birds were randomly assigned to the treatments. The birds were fed corn-soybean meal as a source of fat. Feed and water were both provided ad libitum. The treatments are shown in Table 11. Except for Maxiban (MAX), administered through the feed, all other additives were administered through a continuous provision of water. The water additives were Quillaia saponaria extract or Q. saponaria extract, combined with oregano oil. No birds were replaced during the study.

TABLE 13
Experimental design

| | | | E. maxima | CP challenge |
|---|---|---|---|---|
| | | | challenge | (Days 19, |
| Treatment | | Inclusion | (Day 14) | 20, and 21) |
| Control | CON | — | — | — |
| Control/challenge | POS | — | Yes | Yes |
| Maxiban | MÁX. | | Yes | Yes |
| Quillaia-oregano | QOE1 | 0.12 g/liter | Yes | Yes |
| emulsion | | | | |
| Quillaia-oregano | QOE2 | 0.30 g/liter | Yes | Yes |
| emulsion | | | | |
| Liquid Quillaia | QLS | 0.06 g/liter | Yes | Yes |
| solution | | | | |

On day 14, all the birds (except for CON), were, individually, orally inoculated ˜5000 Eimeria maxima oocysts per 1 mL dose. All the birds in the CON group were administered 1 mL of distilled water. From day 19 to day 21, all the birds, except for CON, were given an inoculum of ˜108 cfu/ml of C. perfringens (CP) in culture broth. The CP was an isolate from a clinical case of necrotic enteritis (NE).

Sample description: QOE1 is a mixture of Quillaia extract, polymer and an oregano emulsion which are mixed by homogenization at 3000 rpm for 2 hours. QOE2 is a mixture of Quillaia extract, oregano oil, Tocopherol, Glycerin and polymer by way of a high shear homogenization process at 3000-5000 rpm for 10 minutes, followed by high pressure at 300-500 psi in two stages. QLS is a 6% saponin Quillaia water extract.

On day 21, 3 birds/cage were slaughtered and necrotic enteritis lesion score was evaluated using a 0 to 3 scoring scale, 0 being normal, and 3 being the most severe. Feed intake (FI), body weight gain (BWG) and feed conversion ratio (FCR) were recorded every 7 days. Blood samples were taken for (fluorescein isothiocyanate-dextran (FITC-d) analysis on day 21 (2 birds/cage). To this effect, each bird was administered a 1 mL of FITC-D solution (2.2 mg/mL concentration) by gavage, the gavage for each treatment being started after a 2-hour period. Finally, 1 mL of blood is taken, coagulant free, and stored at (4° C. (39.2!° F.), until analysis.

### Results

For the 0 to 21 day period, BWG and FCR in the POS group were significantly different from that in the CON group; 19% FCR increase, and 21% BWG reduction (Table 14). As compared to untreated/infected (POS) birds, the QLS treatment showed a significant FCR reduction (12%) and a 16% BWG increase, where it was similar to the MAX treatment. BWG and FCR did not differ significantly among the MAX, QOE1, QOE2 and QLS groups. The NE challenge had a significant impact on performance, as observed by the differences in BWG and FCR values in the POS group versus the CON group, for the 14 to 21-day period; the birds in the POS group showed a 35% BWG reduction, and a 38% FCR increase versus CON. With respect to untreated/infected (POS) birds, the QLS group showed a significant reduction (16%) and increase (24%) in BWG and FCR, respectively. QOE1 and QOE2 showed a significantly lower FCR than the POS group. The QLS and MAX treatments showed the lowest FCR and highest BWG of all treatments, although they did not significantly differ from the QOE1 and QOE2 treatments. Feed intake was greater in unchallenged birds, both on days 0-21 and days 14-21. Feed intake was slightly different between treatments in the overall study and the period during the challenge (14-20 days).

TABLE 14
Productive parameters - 21 days and 14-21 days

| | Day 0-21 | | | Day 14-21 | | |
|---|---|---|---|---|---|---|
| | FI | BWG | | FI | BWG | |
| Treatment | (kg/cage) | (kg) | FCR | (kg/cage) | (kg) | FCR |
| CON | 6.894a | 0.499a | 1.806cd | 3.417a | 0.292a | 1.505e |
| POS | 6.476ab | 0.393e | 2.142a | 3.122abc | 0.191e | 2.076a |
| MÁX. | 6.177b | 0.423cde | 1.914 ac | 2.942c | 0.240bc | 1.576de |
| QOE1 | 6.455ab | 0.436cde | 1.911 ac | 3.025bc | 0.214cde | 1.795 ac |
| QOE2 | 6.375ab | 0.440cd | 1.860cd | 2.996 ac | 0.209de | 1.801 ac |
| QLS | 6.658ab | 0.454bc | 1.887c | 3.235abc | 0.236bcd | 1.753cd |

Within a column, the values with different letters are significantly different (P < 0.05).

During entire trial period (0-28 days), all the groups showed a significantly lower FCR than the POS group (Table 15). The POS group had a 23% lower BWG and a 21% FCR increase with respect to the CON group. The QLS treated birds showed a significantly lower FCR than the POS group and a BWG close to that of the unchallenged birds (CON). FCR did not significantly differ between the MAX, QOE1, QOE2 and QLS treatments. BWG was similar between POS, MAX, QOE1, QOE2. However, BWG was significantly higher in the QLS treatment versus POS, MAX and QOE1.

Following the challenge, during the period of 14-28 day period, the effect of challenge on the birds was clear, as observed by the significantly lower BGW and higher FCR of the POS versus CON group (Table 15). The birds in QOE1, QOE2 and MAX treatments had significantly lower BWG than CON. BWG did not significantly differ between the CON and QLS groups. There was a remarkable difference in the BWG between the QLS and QOE2 groups versus the POS group, showing a 39% and 14% increase, respectively. FCR did not differ significantly between the QOE1, QOE2, and QLS treatments, but all were higher than CON and MAX. Except for the QOE1 treatment, all the treatments showed a significantly lower FCR than POS. In general, FI was similar between POS and other groups; CON showed the highest FI.

TABLE 15
Productive parameters - 0-28 days and 14-28 days

| | Day 0-28 | | | Day 14-28 | | |
|---|---|---|---|---|---|---|
| | FI | BWG | | FI | BWG | |
| Treatment | (kg/cage) | (kg) | FCR | (kg/cage) | (kg) | FCR |
| CON | 8.617a | 0.790a | 1.693c | 5.139a | 0.582a | 1.447c |
| POS | 7.864ab | 0.611b | 2.053a | 4.510ab | 0.410b | 1.963a |
| MÁX. | 7.740b | 0.640b | 1.832b | 4.506ab | 0.457b | 1.573c |
| QOE1 | 7.917ab | 0.649b | 1.922b | 4.48ab | 0.427b | 1.851ab |
| QOE2 | 7.467b | 0.697ab | 1.833b | 4.088b | 0.466b | 1.764b |
| QLS | 7.998ab | 0.783a | 1.865b | 4.575ab | 0.565a | 1.767b |

Within a column, the values with different letters are significantly different (P < 0.05).

A clear treatment effect on the lesion score (Table 16) explains the effects of the different treatments at intestinal level. No lesions were detected in CON on day 21. The lesion score values in the MAX and QLS groups were 51% and 45% lower as compared to the POS treatment, respectively, while the lesions in the QOE1 and QOE2 groups did not differ significantly from those as observed in the POS treatment. FITC-d was 10% lower in the QOE2 group than in the POS group, while QOE1 showed a 9% FITC-d reduction versus POS. In contrast, MAX and QLS showed an FITC-d increase as compared to the POS group. This may suggest that there is greater permeability with these treatments.

TABLE 16
Average lesion score and FITC-d on day 21. Within a parameter,
the letters indicate statistical differences (P < 0.05).

| Treatment | Lesion Score | FITC-d (μg/mL) |
|---|---|---|
| CON | 0.000e | 0.495a |
| POS | 1.669a | 0.576abc |
| MÁX, | 0.816cd | 0.617abc |
| QOE1 | 1.149 ac | 0.522ab |
| QOE2 | 1.372ab | 0.516a |
| QLS | 0.924cd | 0.674bc |

In conclusion, a clear impact of E. maxima and C. perfringens challenge on the birds' production parameters was observed; a 23% BWG reduction was observed in infected birds (POS) versus uninfected birds (CON) over the 21-day period. The administration of saponin-rich extract (QLS) or in combination with oregano at 0.30 g/l (QOE2), improved the BWG by 24% and 12%, respectively, and FCR by 9% and 11%, respectively, with respect to the infected controls (POS) over the 21-day period.

The birds that were administered Maxiban (MAX) or QLS had lower mean lesion score values, namely, 51% and 45%, respectively, as compared to the POS group. The birds that were subjected to the QOE1 or QOE2 treatment improved intestinal integrity, with respect to compared to the birds in the POS group, by 10% and 9%, respectively, as determined by FITC-d on day 21.

### Example for Controlling Histomoniasis in Turkeys.

### Methodology

A total of 200 3-week-old female turkeys were randomly grouped into 9 pens. The groups were: control (−) (CON), control (+) (POS), metronidazole (MED), two 750 PPM QSC-treated groups, and two groups treated with 750 PPM QS15-treated groups. The CON group was not challenged or treated with any product, POS was only inoculated a H. meleagridis suspension, and the MED group was inoculated with the same dose and treated with 30 mg/kg metronidazole per bird/day. The CON group was comprised of 16 young female turkeys, while the remainder comprised 23 each. Table 17 summarizes the bird distribution according to treatment, as well as the appropriate product dosage.

TABLE 17
Bird distribution according to treatment.

| | | | Number of | Number of female |
|---|---|---|---|---|
| Treatment | Infected | Dose | repeats | turkeys/repeats |
| CON | No | — | 1 | 16 |
| POS | Yes | — | 2 | 23 |
| MED | Yes | 30 mg- | 2 | 23 |
| | | day/kg | | |
| QSC | Yes | 750 ppm | 2 | 23 |
| QS15 | Yes | 750 ppm | 2 | 23 |

The treatments were started at 3 weeks of age. The H. meleagridis challenge suspension had approximately 100000 cells/mL and was inoculated at 4 weeks of 10 age. Weight was measured at 3, 6 and 8 weeks of age. Feed intake was estimated at 6 and 8 weeks of age. The young female turkey mortality rate was recorded throughout the trial, and lesions were measured by using a previously standardized lesion scoring at 8 weeks of age. Table 18 shows the treatments for this trial.

TABLE 18
Treatment description

| | Example 1 | Example 2 | Example 3 |
|---|---|---|---|
| | (QS15) | (QSC) | (QHS) |
| Quillaia extract (10.5% saponins) | 30-50 | 30-55 | 25-50 |
| Fiber ((inulin, pectin, amylose, | | 2-10 | 2-10 |
| dextran, chitosan, guar gum) | | | |
| Polymer as an astringent (starch, | 5-25 | 2-5 | 4-12 |
| maltodextrin, guar gum, corn syrup, | | | |
| agar, carboxymethyl cellulose) | | | |
| Water | 65-25 | 66-30 | 69-28 |

Sample description: doses are set to have the same content of saponin per treatment.

QS15 is a mixture of saponin-rich extract to which a highly soluble polymer is added as an astringent that promotes optimal extract drying. The drying conditions are input temperature at 110° C.-170° C. (110° F.-338° F.), and atomization at 50-90%, 20%-40% feed flow

QSC is a mixture of Quillaia extract and natural fiber that is mixed by homogenization at 200 rpm for 2 hours at 40° C.-80° C. (40° F.-176° F.). The pH is set to reach lower values according to the fiber's pka, which promotes fiber expansion and allows the capture/fixation of saponin molecules, forming mixed soluble compounds as triggered by the sugars of the adsorbed saponins. Subsequently, a polymer that is used to promote the formation of a film on the surface of the drops that captures the remaining water and promotes the drying process is added. Finally, mixture's pH of the mixture is set to promote the formation of compounds and improve drying performance. The mixture must be put in the dryer at a suitable temperature allowing fluidity and preventing the formation of caramelized particles in the sprayer.

QHS is a Quillaia extract containing different polymer sources used to create a stable grid that allows it to pass through the intestinal tract and reach the midgut and cecum. Firstly, fibers are added, pH being set to reach lower values according to the fibers' pka, which promotes fiber expansion and allows to capture/fix saponin molecules, forming mixed soluble compounds triggered by the sugars of the adsorbed saponins. Subsequently, different low-soluble polymers and intestinal degradation, which are basically used to generate a more resistant film on the surface of the particles during the drying process, are added.

Drying conditions are adapted to the polymeric matrix where the most important variables are input temperature, (150° C.-200° C. (302° F.-392° F.), 40%-70% atomization, and 10%-30% feed flow

### Results

Strain 14810, used in the present study, had previously shown to reproduce the Histomoniasis condition in turkeys, with lesions in the cecum and liver.

Table 19 shows the mortality rates and cecum and liver lesion score. The CON group showed no mortality throughout the trial, thus validating the methodology implemented. The MED group only showed 2% mortality due to Histomoniasis. The POS, QSC and QS15 groups showed female turkey mortality rates of 51%, 38% and 63%, respectively, the latter two values being statistically different from each other (p<0.05). The above shows that the polymers used in the QS15 treatment do not generate a highly resistant film against gastrointestinal factors allowing the appropriate doses of saponin to reach the cecum; therefore, the saponins are easily released.

TABLE 19
Total mortality rate by treated group,
cecum and liver lesion scoring

| Treatment | CON | POS | MED | QSC | QS15 |
|---|---|---|---|---|---|
| Mortality (%) | 0a | 51bc | 2a | 38b | 63c |
| Liver lesion score | 0a | 1.41c | 0.0a | 0.8bc | 1.67c |
| Cecum lesion score | 0a | 2.29bc | 0.2a | 1.64b | 2.65c |

71% of the turkeys that died of Histomoniasis took place between 13 and 15 days post inoculation (d.p.i.). Table 19 shows the average lesion scores for cecum between the different groups. No statistically significant differences (p<0.05) could be observed using a non-parametric test between the QS15 and POS groups, or the latter with QSC. However, lower lesion intensity in the QSC group could be observed.

As for the lesions observed in the liver of birds having died of Histomoniasis and slaughtered, in general, the lesion intensity observed was comparatively lower for the cecum. However, the same relationship remained between the different groups, the lesion intensity recorded in the QSC group being statistically lower with respect to the QS15 group.

### Example for Controlling Histomoniasis in Turkeys

### Methodology:

A total of 160 3-week-old female turkeys were randomly grouped into 10 pens. The groups were: control (−) CON, control (+) POS, metronidazole (MED), two groups treated with QSC at 1500 ppm (HD greater dose than in the previous trial). The CON group was not challenged or treated with any product; POS control was only inoculated with a H. meleagridis suspension, and the MED group was inoculated with the same dose and treated with metronidazole, 30 mg/kg bird/day. Each described group had a duplicate, and 20 young female turkeys were held in each pen. Table 1208 summarizes bird distribution according to treatments, as well as the appropriate product dosage.

TABLE 20
Bird distribution according to treatments.

| | | | No of | No of female |
|---|---|---|---|---|
| Treatment | Infected | Dose | repeats | turkeys/repeats |
| CON | No | — | 2 | 20 |
| POS | Yes | — | 2 | 20 |
| MED | Yes | 30 mg- | 2 | 20 |
| | | day/kg | | |
| QSC HD | Yes | 1500 ppm | 2 | 20 |

The treatments were started as of 3 weeks of age. The H. meleagridis challenge suspension was 100000 cell/mL, approximately, and was inoculated at 4 weeks of age, via cloacal route. Female turkey mortality rate was recorded throughout the trial, and lesions were measured by using a previously standardized lesion scoring at 8 weeks of age.

### Results

### Mortality and Survival Analysis

Strain 14810, as used in the present study, had previously shown to reproduce the Histomoniasis symptoms in turkeys, with lesions in the cecum and liver. Bird mortality was recorded between days 6 and 26 post inoculation (p.i.), mostly between days 11 and 14 p.i.

Table 21 shows the mortality rates, cecum and liver lesion scores. The CON group did not show any dead birds throughout the trial, thus validating the methodology implemented. The MED group showed a 10% mortality rate, which took place later than the other groups. The POS and QSC HD groups showed female turkey mortality rates of 50% and 35%, respectively, with no statistically different values as identified between them (p≥0.05).

TABLE 21
Total mortality rate by treated group, cecum and liver lesion score.

| Treatment | CON | POS | MED | QSC HD |
|---|---|---|---|---|
| Mortality (%) | 0a | 50b | 10a | 35b |
| Liver lesion score | 0a | 1.28c | 0.28ab | 0.9bc |
| Cecum lesion score | 0a | 2.28c | 1.3b | 1.13b |

57% of dead turkeys due to Histomoniasis took place mostly between days 12 and 14 p.i.

Table 21 shows the average cecum lesion score between the different groups. No statistically significant differences (p>0.05) could be observed using non-parametric testing between the QSC HD and POS groups. However, a lower lesion intensity in the QSC-HD group was actually shown, which was still slightly better at reducing lesions than Metronidazole in the cecum.

In general, the lesion intensity and frequency observed in the liver of birds having died of Histomoniasis, and slaughtered, was lower as compared to the cecum. In this case, QSC HD could not reduce lesion intensity with respect to MED, but it did show a decrease as compared to POS.

### Example for Controlling Histomoniasis in Turkeys

### Methodology:

A total of 160 3-week-old female turkeys were randomly grouped into 10 pens. The groups were: control (−) CON, control (+) POS, MED (metronidazole), and two different treatments, (QSC and QHS's active content under treatment) is set). Control group (−) was not challenged or treated with any product, control (+) was only inoculated with a H. meleagridis suspension, and the MED group was inoculated with the same dose and treated with metronidazole, 30 mg/kg bird/day. Each group described had a duplicate, except for control groups CON and MED, and 20 young female turkeys were held in each pen. Table 22 summarizes bird distribution according to treatments, as well as the appropriate product dosage.

TABLE 22
Bird distribution according to treatments

| | | | No of | No of female |
|---|---|---|---|---|
| Treatment | Infected | Dose | repeats | turkeys/repeats |
| CON | No | — | 1 | 20 |
| POS | Yes | — | 2 | 20 |
| MED | Yes | 30 mg- | 1 | 20 |
| | | day/kg | | |
| QHS | Yes | 1500 ppm | 2 | 20 |
| QSC | Yes | 750 ppm | 2 | 20 |

The treatments were started as of 3 weeks of age. The H. meleagridis challenge suspension contained 100000 cells/mL, approximately, and was inoculated as of 4 weeks of age, cloacal route. The female turkey mortality rate was recorded throughout the trial, and lesions were measured by using a previously standardized lesion scoring at 8 weeks of age.

### Results

Strain 14810, as used in the present study had previously shown to reproduce the Histomoniasis condition in turkeys, with cecum lesions. The bird mortality was recorded between days 10 and 28, post inoculation (p.i.), mostly between days 12 and 20 p.i.

The mortality rates and cecum and liver lesion scores are shown in Table 23. The CON group did not show any birds dying throughout the trial, thus validating the methodology implemented. The MED group showed a 25% mortality rate, which was significantly lower than that in the POS group, but not in the CON group. QSC and QHS did not show any significant differences between each other, however, only QSC differed from the POS group. Further, the QHS group showed slightly better outcomes than the QSC group. All treatments showed the same level of mortality as the MED group (p=0.05).

TABLE 23
Total mortality rates by treated group,
cecum and liver lesion score.

| Treatment | CON | POS | MED | QSC | QHS |
|---|---|---|---|---|---|
| Mortality (%) | 0a | 65.00d | 25.00ab | 48.00bc | 43.00b |
| Liver lesion score | 0a | 2.1d | 0.3a | 1.0bc | 1.6c |
| Cecum lesion score | 0a | 2.3d | 1.5c | 1.0b | 1.2c |

Cecum lesion score averages between the different groups. All groups showed statistically significant differences (p=0.05) with respect to the POS group, and showed no differences between them using a non-parametric test, except for the CON group. QSC and QHS treatments were equally effective at reducing cecum lesions as compared to Metronidazole. Both, QSC and QHS, manage to reduce lesion intensity as observed in the liver of birds that died of Histomoniasis and were slaughtered as compared to the POS group, but are not as effective as Metronidazole at reducing the lesions.

### Example for Controlling Histomoniasis in Turkeys.

A total of 200 3-week-old young female turkeys were randomly grouped in 10 pens. The groups were: control (−) CON, control (+) POS, MED (metronidazole), 3 groups treated with BQ. BQ is a quillay wood-based additive where three different batches were evaluated with (2.5-5.5%) saponins. The CON control group was not challenged or treated with any product, the POS control was only inoculated with a H. meleagridis suspension, and the MED group was inoculated with the same dose and treated with (30 mg/kg metronidazole per bird/day. All the BQ groups were treated with 3250 ppm from the respective product batch evaluated. Each group described had a duplicate, except for the CON and MED groups, and 20 young female turkeys were held in each of pen. Table 24 summarizes bird distribution according to treatments, as well as the appropriate product dosage.

TABLE 24
Experimental design with bird distribution.

| | | | No of | No of female |
|---|---|---|---|---|
| Treatment | Infected | Dose | repeats | turkeys/repeats |
| CON | No | — | 2 | 20 |
| POS | Yes | — | 2 | 20 |
| MED | Yes | 30 mg- | 2 | 20 |
| | | day/kg | | |
| BQ-1 | Yes | 3250 ppm | 2 | 20 |
| BQ-2 | Yes | 3250 ppm | 2 | 20 |
| BQ-3 | Yes | 3250 ppm | 2 | 20 |

The treatments were started as of 3 weeks of age. The H. meleagridis challenge suspension contained 100000 cell/ml, approximately, and was inoculated at 4 weeks of age, via cloacal route. The female turkey mortality rate was recorded throughout the trial, and lesions were measured by using a previously standardized lesion scoring at 8 weeks of age.

### Results

Hm strain 14810, as used in the present study, had previously shown to reproduce the symptoms of Histomoniasis in turkeys, with lesions in the cecum and liver. The bird mortality was recorded between days 12 and 22 p.i. mostly between days 14 and 17 p.i. (post inoculation). The mortality rates, and the cecum and liver lesion scores are shown in Table 25. The CON group did not show any dead birds throughout the trial, thus validating the methodology implemented. The MED group showed a 10% mortality rate, which was significantly lower than hat in the POS group, but not the in the CON group. The three BQ groups showed no significant differences between themselves or from the POS group, although group 3 showed better performance, as its mortality was statistically equal to the MED group (p=0.05).

TABLE 25
Total mortality rates by treated group,
cecum and liver lesion score.

| Treatment | CON | POS | MED | BQ-1 | BQ-2 | BQ-3 |
|---|---|---|---|---|---|---|
| Mortality | 0a | 45.00c | 10.00ab | 35.00c | 35.00c | 28.00bc |
| (%) | | | | | | |
| Liver | 0a | 1b | 0.5ab | 0.53b | 0.8b | 0.5b |
| lesion | | | | | | |
| score | | | | | | |
| Cecum | 0a | 1.43b | 0.85a | 1.25b | 1.1b | 0.93ab |
| lesion | | | | | | |
| score | | | | | | |

The average lesion score for the cecum among the different groups shows that there were no significant differences between the different BQ batches, but the BQ-3 product showed a lower value, and was also the only one capable of showing values similar to the MED and CON groups. The above shows the great variability of a mainly pulverized wood-based product, as its active ingredients that exert the effect at intestinal level are not standardized. What is interesting is that wood contains simple sugars and polymers cellulose, hemicellulose, proteins, which also delay the release, but cannot modulate it. Except for the CON group, no other treatment showed statistically significant differences (p=0.05) with respect to the POS group in the lesions observed in the liver of birds that died due to Histomoniasis and slaughtered.

### Example 5 for Controlling Histomoniasis in Turkeys

A total of 290 3-week-old female turkeys were randomly grouped in 13 pens. The groups were: control (−) CON, control (+) POS, two BQ treated groups from the same batches described in Table 21, but at a higher dose than the previous trial. The CON control group was not challenged or treated with any product, the POS control was only inoculated with a H. meleagridis suspension. All BQ-1 and BQ-2 groups were treated with 3750 ppm of the respective product. Each group described showed four repeats, except for the CON control group. Table 26 summarizes the bird distribution according to treatments, as well as the appropriate product dosage.

TABLE 26
Bird distribution according to treatments.

| | | | No of | No of female |
|---|---|---|---|---|
| Treatment | Infected | Dose | repeats | turkeys/repeats |
| CON | No | — | 1 | 14 |
| POS | Yes | — | 4 | 23 |
| BQ-1 | Yes | 3750 ppm | 4 | 23 |
| BQ-2 | Yes | 3750 ppm | 4 | 23 |

The treatments started to be administered at 3 weeks of age. The H. meleagridis challenge suspension had approximately 100000 cells/mL and was inoculated at 4 weeks of age, via cloacal route. The female turkeys mortality was recorded throughout the trial, and lesions were measured using a previously standardized lesion scoring at 8 weeks of age.

### Results

The Hm strain 14810, as used in the present study, had previously shown to reproduce the symptoms of Histomoniasis in turkeys, with cecum and liver lesions. The bird mortality was recorded between days 11 and 27 post inoculation (p.i.), mostly between days 14 and 17 (post inoculation (p.i.)

The female turkeys mortality rates are summarized in Table 27. The CON group did not show any dead birds throughout the trial, thus validating the methodology implemented. The POS group showed a 54% mortality rate. The three BQ-1 group showed a 58% rate, and the BQ-2 a 62% rate. These three groups did not show any statistically different values significant differences between each other (p=0.05).

TABLE 27
Total mortality rate by treated group, cecum and liver lesion score.

| Treatment | CON | POS | BQ-1 | BQ-2 |
|---|---|---|---|---|
| Mortality (%) | 0a | 54.00b | 58.00b | 62.00b |
| Liver lesion score | 0a | 1.16b | 1.18b | 0.92b |
| Cecum lesion score | 0a | 1.84b | 1..92b | 1.74b |

As for scores, the differences observed in the groups treated with the products did not show any different values from those in the POS group. This shows that the use of this product is not adequate for controlling Histomoniasis, since it shows high variability and the release cannot be modulated.

### Example for Controlling Histomoniasis in Turkeys

A total of 281 3-week-old young female turkeys were randomly grouped in 13 pens. The groups were: control (−) CON, control (+) POS, a group treated with BQ-1, another group treated with the QHS sample, and a last group having been treated with Q-OR. The control group was not challenged or treated with any product; the POS control was only inoculated with an H. meleagridis suspension. Each group described showed three repeats, except for the CON control group and the POS control. Table 28 summarizes the bird distribution according to treatments, as well as the appropriate product dosage.

TABLE 28
Bird distribution according to treatments.

| | | | No of | No of female |
|---|---|---|---|---|
| Treatment | Infected | Dose | repeats | turkeys/repeats |
| CON | No | — | 1 | 17 |
| POS | Yes | — | 2 | 22 |
| BQ-1 | Yes | 3250 ppm | 3 | 22 |
| QHS | Yes | 1085 ppm | 3 | 22 |
| Q-OR | Yes | 500 ppm | 3 | 22 |

The treatments with botanical extracts stated to be administered in the feed, at 3 weeks of age. The H. meleagridis challenge suspension (contained 100000 cells/ml, approximately, and was inoculated at 3 weeks of age, via cloacal route. The female turkeys mortality rate was recorded throughout the trial, and the lesions were measured applying a previously standardized lesion scoring in all the dead birds accruing during the trial, plus the observations of the final slaughter.

### Sample Description:

BQ-1: is an additive that is mainly based on (quillay wood saponins between (2.5-5.5%).

QHS is a Quillaia extract containing different polymer sources used to create a stable grid that allows it to go through the intestinal tract and reach the midgut and cecum. Firstly, fibers are added, the pH is set to reach lower values according to the fibers' pka, which promotes fiber expansion and allows the fibers to capture/fix saponin molecules, forming mixed soluble compounds triggered by the sugars of the adsorbed saponins. Subsequently, different polymers having low solubility and intestinal degradation, which are basically used to generate a more resistant film on the surface of the particles during the drying process, are added.

Q-OR is a powdered emulsion containing oregano essential oil (2-30%), antioxidants as rancidity stabilizing agents (0.1-5%), highly soluble polymers as a drying agent (10-50%) and saponin-rich extract (0.1-20%).

### Results

### Mortality and Survival Analysis

Histomoniasis strain 14810, as used in the present study had previously shown to reproduce Histomoniasis symptoms in turkeys, with cecum and liver lesions. Bird mortality was recorded from day 11 post inoculation (p.i.) to day 26 p.i.

The mortality rates and cecum and liver lesion score are shown in Table 29. The CON group showed no mortality throughout the trial, thus validating the methodology implemented.

The POS group showed a accrued mortality rate of 43%, while the BQ-1, QHS, and Q-OR treatment groups showed 8%, 27%, and 44%, respectively. The BQ-1 and QHS treatments showed significantly decrease mortality rates compared to that in the POS group, but were also different from each other. The Q-OR treatment failed to decrease its mortality rate significantly with respect to the POS group (p=0.05).

TABLE 29
Total mortality rate by treated group, cecum and liver lesion score.

| Treatment | CON | POS | BQ-1 | QHS | Q-OR |
|---|---|---|---|---|---|
| Mortality (%) | 0a | 43.00d | 8.00b | 27.00c | 44.00d |
| Liver lesion score | 0a | 1.93c | 0.32a | 1b | 1.17b |
| Cecum lesion score | 0a | 1.61c | 0.29a | 0.7ab | 1.18bc |

The average cecum lesion scores between groups are summarized regarding both birds that died during the trial and birds that were slaughtered (at trial's end. The Q-OR sample proved to be no different from the POS group, whereas the BQ-1 sample showed similar results with respect to the CON group, but it was also not statistically different from the QHS group (p=0.05). The QHS and Q-OR groups showed a reduction in the severity of the lesions found in the liver of birds killed by Histomoniasis with respect to the POS group. Both groups (QHS and BQ-1), as compared to the POS group, did not show any differences between each other using a non-parametric test. The group treated with the BQ-1 product showed a decrease in the severity of the clinical condition due to Histomoniasis, as compared to the POS group. On the other hand, the group treated with the QHS product also showed good results, showing a mortality rate drop and attenuating, in a statistically significant manner, the slope of the survival curve with respect to the POS group.

Even though BQ-1 proved to be the best treatment in this trial, its variability is due to its composition, which does not make it viable for controlling Histomoniasis, since its results are not consistent between the different trials (Tables 25, 27, and 29).

Finally, the group treated with the Q-OR sample lacked the capacity to fight off Histomoniasis, failing to be different from the POS group as per the parameters described.

We claim:

1. A concentrated, liquid and/or powdered coated composition to control digestive tract diseases in animals, comprising:
between 0.15-95% w/w saponin-rich (or pulverized wood extract;
between 0.1%-35% w/w of fiber; and
entre un 0-35% w/w of polymer; and
water sq for 100%,
in one of the embodiments of the present invention, the size of the particles in the coated concentrated composition is between 1-10000 μm.

2. The composition, according to claim 1, wherein it optionally comprises particles containing further actives to transmit the composition through the micro-emulsion systems.

3. The composition, according to claim 2, wherein the further actives to transmit through the micro-emulsion systems may comprise:
optionally, between 5%-30% w/w of a vegetal essential oil;
optionally, between 5%-30% w/w of a co-surfactant;
optionally, between 0.1%-5% w/w of a natural, vegetal, antioxidant-rich extract; and/or
optionally, between 0%-20% w/w of an emulsifying agent.

4. The composition, according to claim 1, wherein the saponin-rich extract is selected from quillay (soap bark), yucca, quinoa, fenugreek, garlic, fennel tea, ginseng, alfalfa, oats, fique, chocho, cabuya (agave), black bean, or combinations thereof.

5. The composition according to claim 1, wherein the polymer is selected from among modified starch, starch, maltodextrin, guar gum, corn syrup, agar, proteins, alginate, or combinations thereof.

6. The composition, according to claim 1, wherein the fiber is selected from among carboxymethylcellulose, guar gum, pectine, xanthan gum, inulin, amylose, dextran, chitosan, or combinations thereof.

7. The composition, according to claim 1, wherein the vegetal essential oil is selected from among oregano essential oil, thyme, lemongrass, eucalyptus, orange, clove, bayleaf, lemon, cinnamon, mustard, savory, copaiba, or combinations thereof.

8. The composition, according to claim 1, wherein the antioxidant agent is selected from among rosemary, Tocopherol, curcumina, asthaxantin, or combinations thereof.

9. The composition, according to claim 1, wherein the co-solvent agent of the composition is elected from among polyols, glycerin, propylene glycol, or combinations thereof.

10. The composition, according to claim 1, wherein the emulsifying agent is selected from among saponins, lecithins, polar lipids, or combinations thereof.

11. The composition, according to claim 1, wherein it is active against pathogens causing cecum diseases or other intestinal diseases of interest in animal health, or combinations thereof.

12. The composition, according to claim 1, wherein it may be orally administered to the animal through pills, capsules, injection, in the feed, powdered, or in liquid state, or combinations thereof.

13. The composition, according to claim 1, wherein it is optionally powdered, where the composition is dried by atomization processes, fluidized bedand7or tray drying, under temperatures between 120° C.-200° C. (248° F.-392° F.) which depend on the combination of polymers used.

14. Use of the coated concentrated composition, according to claim 1, wherein it is useful to prepare an additive to control gastrointestinal infections in animals and improve the intestinal health in animals.

15. The use in claim 14, wherein it is useful to prepare an additive to control gastrointestinal infections in in vitro and in vivo conditions.

16. A procedure for preparing a liquid, coated concentrated composition to control digestive tract diseases in animals, comprising the following steps:
a) mixing an saponin-rich extract with oregano that is transmitted via micro-emulsion technology;
b) the micro-emulsions being prepared following the steps of:
i) preparing the continuous phase by way of mechanical stirring, containing an saponin-rich extract as emulsifying agents, glycerin as co-solvents, polymers as a stabilizer, and water as a solvent;
ii) Preparing the lipidic stage containing an essential oil and a mixture of natural antioxidants at a emulsifying agent:oil rate of 1:2-1:10;
iii) Mixing both stages through a high shear homogenization process to form the pre-emulsion; and
iv) Homogenizing by using two-stage high pressure equipment.

17. A procedure for preparing a power-coated concentrated composition to control digestive tract diseases in animals comprising the following steps:
a) mixing the saponin-rich extract and natural fibers through mechanical stirring and temperature control until reaching a homogenous mixture;
b) setting pH to reach lower values depending on pKa of the fibers used for complete gelification and capture of the saponins in the grid by way of interactions between them and the fiber;
c) adding the fiber and saponins at 1:1 ratio, more specifically, at 1:10, more specifically, at 1:50, which is key to the formation of particles;
d) adding the polymer, under constant stirring, as a astringent;
d) Increasing pH to promote the formation of the capsule and drying's good performance;
e) keeping mix temperature constant for maintaining mixture's necessary fluidity necessary for the drying process; and
f) drying the mixture by atomization, keeping a constant delta between the dryer's input temperature.
